(19) United States
(12) Patent Application Publication
(10) Pub. No.: US 2005/0176136 A1
(43) Pub. Date: Aug. 11, 2005

(54) AFINITY DOMAIN FOR ANALYTE SENSOR

(75) Inventors: John Burd, San Diego, CA (US); Mark Tapsak, Orangeville, PA (US); Rathbun Rhodes, Madison, WI (US)

Correspondence Address: KNOBBE MARTENS OLSON & BEAR LLP, 2040 MAIN STREET, FOURTEENTH FLOOR, IRVINE, CA, 92614, US

(73) Assignee: DexCom, Inc., San Diego, CA (US)

(21) Appl. No.: 10/991,353

(22) Filed: Nov. 16, 2004

Related U.S. Application Data
(60) Provisional application No. 60/523,832, filed on Nov. 19, 2003.
(60) Provisional application No. 60/587,787, filed on Jul. 13, 2004.
(60) Provisional application No. 60/614,683, filed on Sep. 30, 2004.

(51) Int. Cl. C12M 1/34; A61B 5/00

(52) U.S. Cl. 435/287.2; 600/315

(57) ABSTRACT

Abstract of the Disclosure

The preferred embodiments provide a membrane system, particularly for use on an electrochemical sensor, wherein the membrane system includes an affinity domain that dampens the effects of target interferant(s) on the sensor. The affinity domain can be layer, surface, region, and/or portion of the membrane system formed using sorbents that have an affinity for the target interferant. The sorbents can be adapted to adsorb the interferants, for example using adsorbents such as chromatography packing materials. The sorbents can also be adapted to absorb the interferants by imprinting a molecular structure on the material that forms the affinity domain such that target interferants bind to the imprinted surfaces at the molecular level.

### Detailed Description of the Invention

### RELATED APPLICATIONS

This application claims the benefit of priority under 35 U.S.C. § 119(e) to U.S. Provisional Application No. 60/523,832 filed November 19, 2003, U.S. Provisional Application No. 60/587,787, filed July 13, 2004, and U.S. Provisional Application No. 60/614,683, filed September 30, 2004. Each above-referenced prior application is incorporated by reference herein in its entirety.

### FIELD OF THE INVENTION

The present invention relates generally to systems and methods involving the detection or measurement of analytes. More particularly, the present invention relates to reducing the effects of interfering species on a signal obtained from a glucose sensor.

### BACKGROUND OF THE INVENTION

A variety of sensors are known that use an electrochemical cell to provide output signals by which the presence or absence of an analyte in a sample can be determined. For example in an electrochemical cell, an analyte (or a species derived from it) that is electroactive generates a detectable signal at an electrode, and this signal can be used to detect or measure the presence and/or amount within a biological sample. In some conventional sensors, an enzyme is provided that reacts with the analyte to be measured, and the byproduct of the reaction is quantified at the electrode. An enzyme has the advantage that it can be very specific to an analyte and also, when the analyte itself is not sufficiently electroactive, can be used to interact with the analyte to generate another species which is electroactive and to which the sensor can produce a desired output. In one conventional amperometric glucose oxidase-based glucose sensor, immobilized glucose oxidase catalyses the oxidation of glucose to form hydrogen peroxide, which is then quantified by amperometric measurement (e.g., increase in electrical current) at a polarized electrode.

One problem with such sensors is that they may detect other electroactive species that are not intentionally being measured (e.g., interfering species.) This causes an increase in signal strength due to the interfering species. In other words, interfering species can be compounds with an oxidation potential that overlaps with the analyte to be measured (or by product of the enzymatic reaction with the analyte). For example, in a conventional amperometric glucose oxidase-based glucose sensor, interfering species such as acetaminophen, ascorbate, and urea, are known to produce inaccurate signal strength when they are not properly controlled. Similar problems have been seen in other sensor types, for example optical techniques.

Some glucose sensors utilize a membrane system that blocks at least some selected interfering species, such as ascorbate and urea. In some such examples, at least one layer of the membrane system includes a porous structure that has a relatively impermeable matrix with a plurality of "micro holes" or pores of molecular dimensions, such that transfer through these materials is primarily due to passage of species through the pores (e.g., the layer acts as a microporous barrier or sieve block interfering species of a particular size). In other such examples, at least one layer of a membrane system defines a permeability that allows selective dissolution and diffusion of species as a solute through the layer. Unfortunately, it is difficult to find membranes that are satisfactory or reliable in use, especially in vivo, which effectively block all interfering species.

### SUMMARY OF THE INVENTION

Because of the limitations found in the prior art, there is a need for an improvement that is able to reduce the effects of all interfering species, even species that are deemed difficult to eliminate, on a sensor signal.

Accordingly, in a first embodiment, a membrane suitable for use with an analyte sensor is provided, the membrane comprising an affinity domain, wherein the affinity domain comprises a sorbent having an affinity for an interfering species.

In an aspect of the first embodiment, the sorbent has an affinity for a phenol-containing species.

In an aspect of the first embodiment, the sorbent has an affinity for acetaminophen.

In an aspect of the first embodiment, the sorbent comprises an adsorbent substance.

In an aspect of the first embodiment, the adsorbent substance comprises a chromatography-packing material.

In an aspect of the first embodiment, the sorbent comprises a molecularly imprinted surface adapted to bind with the interfering species by covalent adherence.

In an aspect of the first embodiment, the sorbent comprises a molecular structure that has a geometric structure and hydrogen binding capability, wherein the molecular structure is adapted to bind with the interfering species.

In a second embodiment, an electrochemical sensor comprising the membrane of the first embodiment is provided.

In a third embodiment, a wholly implantable glucose sensor comprising the membrane of the first embodiment is provided.

In a fourth embodiment, a transcutaneous glucose sensor comprising the membrane of the first embodiment is provided.

In a fifth embodiment, an analyte sensor for measuring the concentration of an analyte in a host is provided, the sensor comprising a sensing region for sensing the analyte; and a membrane system comprising an affinity domain, the affinity domain having an affinity for an interfering species, wherein the membrane system is disposed adjacent to the sensing region.

In an aspect of the fifth embodiment, the sensing region comprises an electroactive surface and wherein the membrane system comprises an enzyme capable of reacting with the analyte.

In an aspect of the fifth embodiment, the affinity domain comprises a sorbent, wherein the sorbent is configured to slow the diffusion of the interfering species through the membrane system to the sensing region.

In an aspect of the fifth embodiment, the sorbent has an affinity for a phenol-containing species.

In an aspect of the fifth embodiment, the sorbent has an affinity for acetaminophen.

In an aspect of the fifth embodiment, the sensor is adapted for implantation in a soft tissue of the host.

In an aspect of the fifth embodiment, the sensor is adapted for whole implantation within the host.

In an aspect of the fifth embodiment, the sensor is adapted for transcutaneous implantation in the host.

### BRIEF DESCRIPTION OF THE DRAWINGS

Fig. 1A is a perspective view of an implantable glucose sensor 10a in one exemplary embodiment, showing a body, an electrode system, and a membrane system incorporated thereon.

Fig. 1B is a perspective view of an in vivo portion of a transcutaneous glucose sensor in one exemplary embodiment.

Fig. 1C is an illustration that represents a method of forming the sensing membrane in one embodiment.

Fig. 1D is a schematic side view of the sensing membrane in one embodiment.

Fig. 2 is a graph of interferant concentration (relative) versus time (relative), which illustrates the rise and fall of a transient interferant concentration exposed to a sensor in a host’s body.

Fig. 3 is a graph of glucose and acetaminophen concentration versus time, which shows the results from increasing addition of acetaminophen in two test membranes having affinity domains of the preferred embodiments and two control membranes without affinity domains of the preferred embodiments.

### DETAILED DESCRIPTION OF THE PREFERRED EMBODIMENT

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

### Definitions

In order to facilitate an understanding of the preferred embodiment, a number of terms are defined below.

The term "interferant" and "interfering species," as used herein, are broad terms and are used in their ordinary sense, including, without limitation, species that interfere with the measurement of an analyte of interest in a sensor to produce a signal that does not accurately represent the analyte measurement. In one example of an electrochemical sensor, interfering species are compounds with oxidation potentials that overlap with the analyte to be measured.

The term "domain" as used herein is a broad term and is used in its ordinary sense, including, without limitation, regions of the biocompatible membrane that can include layers, uniform or non-uniform gradients (for example, anisotropic), functional aspects of a material, or provided as portions of the membrane.

The term "host" as used herein is a broad term and is used in its ordinary sense, including, without limitation, mammals, particularly humans.

The phrase "continuous (or continual) analyte sensing" as used herein is a broad term and is used in its ordinary sense, including, without limitation, the monitoring of an analyte concentration continuously, continually, and or intermittently (regularly or irregularly), for example, from about less than a second to about every 10 minutes or more.

The term "sensing region" as used herein is a broad term and is used in its ordinary sense, including, without limitation, the region of a monitoring device responsible for the detection of a particular analyte. In one embodiment, the sensing region generally comprises a non-conductive body, a working electrode (anode), a reference electrode and a counter electrode (cathode) passing through and secured within the body forming an electrochemically reactive surface at one location on the body and an electronic connective means at another location on the body, and a membrane system affixed to the body and covering the electrochemically reactive surface. During general operation of the sensor a biological sample (for example, blood or interstitial fluid) or a portion thereof contacts (directly or after passage through one or more membranes or domains) an enzyme (for example, glucose oxidase); the reaction of the biological sample (or portion thereof) results in the formation of reaction products that allow a determination of the analyte (for example, glucose) level in the biological sample. In some embodiments, the membrane system further comprises an enzyme domain (for example, an enzyme layer), and an electrolyte phase (namely, a free-flowing liquid phase comprising an electrolyte-containing fluid described further below). However, the term is sufficiently broad so as to encompass a variety of sensing techniques, for example, enzymatic, chemical, physical, optical, electrochemical, spectrophotometric, polarimetric, amperometric, calorimetric, radiometric, and the like.

The terms "electrochemically reactive surface" and "electroactive surface" as used herein are broad terms and are used in their ordinary sense, including, without limitation, the surface of an electrode where an electrochemical reaction takes place. In the case of the working electrode, the hydrogen peroxide produced by the enzyme catalyzed reaction of the analyte being detected reacts creating a measurable electronic current (for example, detection of glucose analyte utilizing glucose oxidase produces H₂O₂ peroxide as a by product, H₂O₂ reacts with the surface of the working electrode producing two protons (2H⁺), two electrons (2e⁻) and one molecule of oxygen (O₂) which produces the electronic current being detected). In the case of the counter electrode, a reducible species, for example, O₂ is reduced at the electrode surface in order to balance the current being generated by the working electrode.

The term "high oxygen solubility domain" as used herein is a broad term and is used in its ordinary sense, including, without limitation, a domain composed of a material that has higher oxygen solubility than aqueous media so that it concentrates oxygen from the biological fluid surrounding the biointerface membrane. The domain can then act as an oxygen reservoir during times of minimal oxygen need and has the capacity to provide on demand a higher oxygen gradient to facilitate oxygen transport across the membrane. This enhances function in the enzyme reaction domain and at the counter electrode surface when glucose conversion to hydrogen peroxide in the enzyme domain consumes oxygen from the surrounding domains. Thus, this ability of the high oxygen solubility domain to apply a higher flux of oxygen to critical domains when needed improves overall sensor function. The terms "membrane system" and "membrane" as used herein, are broad terms and are used in their ordinary sense, including, but not limited to, a membrane comprising one or more domains, layers, regions, or portions. The term "sorbent" as used herein, is a broad term and is used in its ordinary sense, including, without limitation, to take up and hold by either adsorption or absorption.

The term "sorb," as used herein, is a broad term and is used in its ordinary sense, including, without limitation, to take up and hold by either adsorption or absorption.

The terms "adsorbent" and "adsorbant" as used herein are broad terms and are used in their ordinary sense, including, without limitation, a substance that collects molecules of another substance on its surface.

The terms "absorbent" and "absorbant" as used herein, are broad terms and are used in their ordinary sense, including, without limitation, a substance that takes in and makes a part of an existent whole.

The term "sol-gel material," as used herein, is a broad term and is used in its ordinary sense, including, without limitation, a material that is prepared using a sol-gel method, for example, preparing specialty metal oxide glasses and ceramics by hydrolyzing a chemical precursor or mixture of chemical precursors that pass sequentially through a solution state and a gel state before being dehydrated to a glass or ceramic. Typically, the chemical precursors are metal alkoxides such as tetraethylorthosilicate.

### Description

The preferred embodiments relate to the use of an analyte-measuring device that measures a concentration of analyte or a substance indicative of the concentration or presence of the analyte. In some embodiments, the analyte-measuring devices measure glucose. In alternative some embodiments, the analyte-measuring devices measure other analytes, for example, oxygen, lactate, cholesterol, amino acids, or the like, as is appreciated by one skilled in the art. In some embodiments, the analyte-measuring device is a continuous device, for example a subcutaneous, transdermal, or intravascular device. In some embodiments, the device can analyze a plurality of intermittent blood samples. In some embodiments, the device can analyze a single blood sample.

Although the preferred embodiments illustrate and describe two examples of electrochemical analyte-measuring devices, the affinity domain of the preferred embodiments can be implemented with a wide variety of known analyte-measuring devices, including chemical, physical, optical, electrochemical, spectrophotometric, polarimetric, amperometric, calorimetric, radiometric, or the like. Some analyte-measuring devices that can benefit from the systems and methods of the preferred embodiments include U.S. Patent No. 5,711,861 to Ward et al., U.S. Patent No. 6,642,015 to Vachon et al., U.S. Patent No. 6,654,625 to Say et al., U.S. Patent No. 6,514,718 to Heller, U.S. Patent No. 6,465,066 to Essenpreis et al., U.S. Patent No. 6,214,185 to Offenbacher et al., U.S. Patent No. 5,310,469 to Cunningham et al., and U.S. Patent No. 5,683,562 to Shaffer et al., for example. All of the above patents are incorporated in their entirety herein by reference and are not inclusive of all applicable analyte-measuring devices; in general, the disclosed embodiments are applicable to a variety of analyte-measuring device configurations.

The analyte-measuring device uses any known method, including invasive, minimally invasive, and non-invasive sensing techniques, to provide an output signal indicative of the concentration of the analyte. The output signal is typically a raw signal that is used to provide a useful value of the analyte to a user, such as a patient or doctor, who can use the device. In one preferred embodiment, the analyte-measuring device measures glucose using an electrochemical cell with a membrane system, such as described with reference to U.S. Patent 6,001,067 and U.S. Published Patent Application 2003/0032874, both of which are incorporated by reference herein in their entirety.

### Exemplary Glucose Sensors

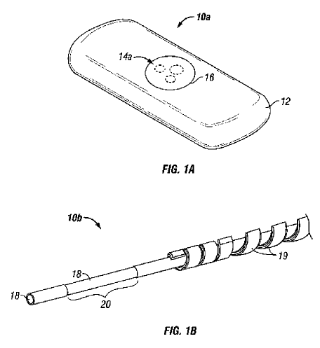

Fig. 1A is a perspective view of an implantable glucose sensor 10a in one exemplary embodiment, showing a body, an electrode system, and a membrane system incorporated thereon. Co-pending U.S. Patent Application 10/838,912, filed May 3, 2004 and entitled "IMPLANTABLE ANALYTE SENSOR," which is incorporated herein by reference in its entirety, describes systems and methods suitable for the implantable glucose sensor of the illustrated embodiment; however, one skilled in the art appreciates a variety of implantable analyte sensors that can benefit from the affinity domain of the preferred embodiments.

The body 12 is preferably formed from epoxy molded around the sensor electronics (not shown), however the body can be formed from a variety of materials, including metals, ceramics, plastics, or composites thereof. Co-pending U.S. Patent Application 10/646,333, entitled, "Optimized Device Geometry for an Implantable Glucose Device" discloses configurations suitable for the body 12, and is incorporated by reference in its entirety.

In one preferred embodiment, the sensor 10a is an enzyme-based sensor, which includes an electrode system 14a (for example, a platinum working electrode, a platinum counter electrode, and a silver/silver chloride reference electrode), which is described in more detail with reference to U.S. Patent Application 09/916,711, entitled "Sensor head for use with implantable devices," which is incorporated herein by reference in its entirety. However a variety of electrode materials and configurations can be used with the implantable glucose sensor of the preferred embodiments. The exposed electroactive surfaces of the electrode system 14a are in contact with an electrolyte phase (not shown), which is a free-flowing fluid phase disposed between a membrane system 16 and the electrode system 14a. The membrane system 16 is deposited over the electroactive surfaces of the electrode system 14a and includes a plurality of domains or layers, such as in more detail below.

In this embodiment, the counter electrode is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction:

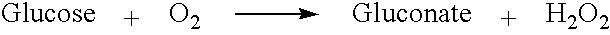

The change in H₂O₂ can be monitored to determine glucose concentration because for each glucose molecule metabolized, there is a proportional change in the product H₂O₂. Oxidation of H₂O₂ by the working electrode is balanced by reduction of ambient oxygen, enzyme generated H₂O₂, or other reducible species at the counter electrode. The H₂O₂ produced from the glucose oxidase reaction further reacts at the surface of working electrode and produces two protons (2H⁺), two electrons (2e⁻), and one oxygen molecule (O₂).

In one embodiment, a potentiostat is employed to monitor the electrochemical reaction at the electroactive surface(s). The potentiostat applies a constant potential to the working and reference electrodes to determine a current value. The current that is produced at the working electrode (and flows through the circuitry to the counter electrode) is substantially proportional to the amount of H₂O₂ that diffuses to the working electrode. Accordingly, a raw signal can be produced that is representative of the concentration of glucose in the user’s body, and therefore can be utilized to estimate a meaningful glucose value.

Fig. 1B is a perspective view of an in vivo portion of a transcutaneous glucose sensor in one exemplary embodiment. Co-pending U.S. Provisional Application 60/587,787, filed July 13, 2004 and U.S. Provisional Application 60/614,683, filed September 30, 2004, describe systems and methods suitable for the transcutaneous glucose sensor of the illustrated embodiment; however, one skilled in the art appreciates a variety of transcutaneous analyte sensors that can benefit from the affinity domain of the preferred embodiments.

In this embodiment, the in vivo portion of the sensor 10b is the portion adapted for insertion under the host’s skin. Preferably, the sensor body 12b is formed from an electrode system comprising two or more electrodes: a working electrode 18 and at least one additional electrode 19, which can function as a counter and/or reference electrode, hereinafter referred to as the reference electrode. Each electrode is formed from a fine wire, with a diameter in the range of 0.001 to 0.010 inches, for example, and can be formed from plated wire or bulk material.

In one embodiment, the working electrode 18 comprises a wire formed from a conductive material, such as platinum, palladium, graphite, gold, carbon, conductive polymer, or the like. The working electrode 18 is configured and arranged to measure the concentration of an analyte. The working electrode 20 is covered with an insulating material, for example a non-conductive polymer. Dip-coating, spray-coating, or other coating or deposition techniques can be used to deposit the insulating material on the working electrode, for example. In one preferred embodiment, the insulating material comprises Parylene, which can be an advantageous conformal coating for its strength, lubricity, and electrical insulation properties, however, a variety of other insulating materials can be used, for example, fluorinated polymers, polyethyleneterephthalate, polyurethane, polyimide, or the like.

The reference electrode 19, which can function as a reference electrode alone, or as a dual reference and counter electrode, is formed from silver, Silver/Silver chloride, or the like. In one embodiment, the reference electrode 19 is formed from a flat wire with rounded edges in order to decrease sharp edges and increase host comfort. Preferably, the reference electrode 19 is juxtapositioned and/or twisted with or around the working electrode 18, however other configurations are also possible. In some embodiments, the reference electrode 19 is helically wound around the working electrode 18 (see Fig. 1B).

The assembly of wires is then optionally coated together with an insulating material, similar to that described above, in order to provide an insulating attachment. Some portion of the coated assembly structure is then stripped, for example using an excimer laser, chemical etching, or the like, to expose the necessary electroactive surfaces. In one implementation, a window 20 is formed on the insulating material to expose an electroactive surface of the working electrode 18 and at least some edges of the sensor are stripped to expose sections of electroactive surface on the reference electrode. Other methods and configurations for exposing electroactive surfaces are also possible, for example by exposing the surfaces of the working electrode 18 between the coils of the reference electrode 19. In some alternative embodiments, additional electrodes can be included within the assembly, for example, a three-electrode system (working, reference, and counter electrodes) and/or including an additional working electrode (which can be used to generate oxygen, configured as a baseline subtracting electrode, or configured for measuring additional analytes, for example).

A membrane system (not shown) is deposited over the electroactive surfaces of the sensor 10b (working electrode and optionally reference electrode) and includes a plurality of domains or layers, such as in more detail below. The membrane system can be deposited using known thin film techniques (for example, spraying, electro-depositing, dipping, or the like). In one exemplary embodiment, each domain is deposited by dipping the sensor into a solution and drawing out the sensor at a speed that provides the appropriate domain thickness. In general, the membrane system can be disposed over (deposited on) the electroactive surfaces using methods appreciated by one skilled in the art.

In the illustrated embodiment, the sensor is a glucose oxidase electrochemical sensor, wherein the working electrode 18 measures the hydrogen peroxide produced by an enzyme catalyzed reaction of the analyte being detected and creates a measurable electronic current (for example, detection of glucose utilizing glucose oxidase produces H₂O₂ peroxide as a by product, H₂O₂ reacts with the surface of the working electrode producing two protons (2H⁺), two electrons (2e⁻) and one molecule of oxygen (O₂) which produces the electronic current being detected), such as described in more detail above and as is appreciated by one skilled in the art.

### Membrane Systems

Preferably, the membrane system 16 described with reference to Figs. 1A and 1B provides one or more of the following functions: 1) support tissue ingrowth and encourage vascularity within the membrane, 2) block to cellular penetration, 3) protection of the exposed electrode surface from the biological environment, 4) diffusion resistance (limitation) of the analyte, 5) a catalyst for enabling an enzymatic reaction, and 6) hydrophilicity at the electrochemically reactive surfaces of the sensor interface, such as described in co-pending U.S. Patent Applications 10/838,912, filed May 3, 2004 and entitled "IMPLANTABLE ANALYTE SENSOR" and 10/885,476, filed July 6, 2004 and entitled "SYSTEMS AND METHODS FOR MANUFACTURE OF AN ANALYTE-MEASURING DEVICE INCLUDING A MEMBRANE SYSTEM" both of which are incorporated herein by reference in their entirety. Accordingly, membrane systems preferably include a plurality of domains or layers, for example, a cell disruptive domain, a cell impermeable domain, a resistance domain, an enzyme domain (for example, glucose oxidase), and an electrolyte domain, and can additionally include a high oxygen solubility domain (not shown), and/or a bioprotective domain (not shown), such as described in more detail in the above-cited U.S. Patent Application No. 10/838,912. However, it is understood that a membrane systems modified for other devices, for example, by including fewer or additional domains is within the scope of the preferred embodiments.

In some embodiments, the membrane system includes an interference domain that blocks some interfering species; such as described in co-pending U.S. Patent Application No. 09/916,711, entitled, "SENSOR HEAD FOR USE WITH IMPLANTABLE DEVICES," which is incorporated herein by reference in its entirety. Membrane systems including an interference domain that can limit diffusion of high molecular weight species have been described in the prior art. The interference domain generally serve to allow analytes and other substances that are to be measured by the electrodes to pass through, while preventing passage of other substances, including interfering species, such as ascorbate and urea. In one exemplary embodiment, the interference domain is constructed from polyurethane and has a thickness of from about 0.1 to 5 microns.

Although in some embodiments, an interference domain does successfully block some interfering species described above, it does not sufficiently block other interfering species, such as acetaminophen, which is a known interferant in many hydrogen peroxide based glucose sensors. 4-Acetaminophenol (4-AAP, common name acetaminophen or paracetamol) is a nonprescription medication useful in the treatment of mild pain or fever, for example, acetaminophen can be found in Tylenol®. Acetaminophen is a common medication, and when ingested, can cause transient, signal artifacts in an electrochemical glucose sensor (see Figs. 1A and 1B, for example). Acetaminophen is only one example of an interferant that can be targeted using the affinity domain of the preferred embodiments, however.

Accordingly, in the preferred embodiments, the membrane system includes a domain that reduces the effects of transient, non-analyte related signal artifacts due to interfering species, and is hereinafter referred to as the "affinity domain." The affinity domain is adapted to sorb interfering species, such as acetaminophen, or the like, to dampen the effects of the interfering species on the signal.

In the preferred embodiments, the domains of the membrane system are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones and block copolymers thereof including, for example, di-block, tri-block, alternating, random and graft copolymers. Co-pending U. S. Patent Application 10/838,912, which is incorporated herein by reference in its entirety, describes biointerface and sensing membrane configurations and materials that can be applied to the preferred embodiments. Fig. 1C is an illustration that represents a method of forming the sensing membrane in one embodiment. Fig. 1D is a schematic side view of the sensing membrane in one embodiment. In this embodiment, the sensing membrane 88 includes a resistance domain 90, an enzyme domain 92, an interference domain 94, and an electrolyte domain 96. Preferably, the domains are serially cast upon a liner 98, and all of the domains are formed on a supporting platform 100; however, in alternative embodiments the membrane domains can be formed directly on the sensing region, for example, by spin-, spray-, or dip-coating. Alternatively, an affinity domain can be included between any layers, or within a layer, in the above-described configuration. While the above-described ordering of layers is generally preferred, other ordering can be desirable in certain embodiments. For example, the location of the interference domain or layer can be the same as that depicted in Figures 1C and 1D, or alternatively, it can be in a different location.

### Affinity Domain

Much of the description of the preferred embodiments focus on providing an affinity domain with an affinity to acetaminophen, which is a known interferant in the art of amperometric glucose sensors because it generates a positive signal independent of glucose concentration. However, the affinity domain of the preferred embodiments can be implemented to include an affinity for numerous other known interferants. For example, optical glucose sensors suffer from interference from species such as triglyceride, albumin, and gamma globulin. In general, the effects of any known interferants on sensor signals can be reduced using the concepts described herein.

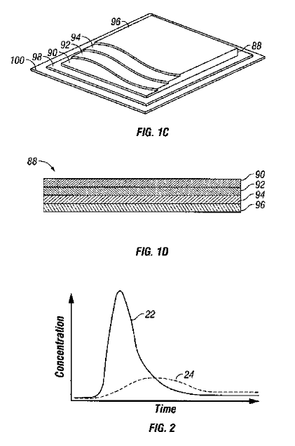

Fig. 2 is a graph of interferant concentration (relative) versus time (relative), which illustrates the rise and fall of a transient interferant concentration exposed to a sensor in a host’s body. For example, when acetaminophen is taken orally, the systemic concentration rises quickly and then decreases rapidly as the species is cleared by the system, such as illustrated in Fig. 2, line 22. Medication such as acetaminophen is typically taken transiently (e.g., rather than continually) and therefore produces transient, non-glucose related signal artifacts on a glucose-measuring device. Because an elevated acetaminophen concentration is a transient event in the host, moderating acetaminophen concentration is generally only required for discrete periods of time.

According to the preferred embodiments, the affinity domain has an "affinity" for the interferant to be blocked, and therefore sorbs that interferant; by sorbing the interferant into the membrane system, the effects on the resulting signal are reduced. The interferant is subsequently released from the affinity domain but at a slower rate, resulting in a lower signal at any point in time. Consequently, the local concentration of interferant presented to the electrochemically reactive surface of the sensor is moderated as illustrated in Fig. 2, line 24.

While not wishing to be bound by any particular theory, it is believed that the area under both curves is substantially equal, however the local concentration of interfering species at the sensor with the affinity domain of the preferred embodiments is sufficiently lowered over time (e.g., line 24), as compared to a membrane system without the affinity domain (e.g., line 22). In other words, the affinity domain of the preferred embodiments slows the diffusion of the interfering species on the signal, such that the signal deviation due to the interferant is below a level that can substantially interfere with sensor accuracy.

The preferred embodiments provide a membrane system, particularly for use on an electrochemical sensor, wherein the membrane system includes an affinity domain. The affinity domain can be layer, surface, region, and/or portion of the membrane system and manufactured using a variety of methods. In general, the affinity domain is formed using sorbents with an affinity for the target interferant(s). Sorbents include any substance (e.g., molecule, particle, coating, or the like) that has a stronger affinity for a particular molecule or compound (e.g., interfering species) than another (e.g., measured analyte or substance). The sorbents of the preferred embodiments provide for the retention of an interfering species, such that the interfering species will be at least temporarily immobilized, and will take a longer time to pass through the affinity domain.

In some embodiments, the sorbents are polymeric adsorbents, such as chromatography-packing materials. The chromatography-packing materials can be selected, modified, or otherwise adapted to possess an affinity for a target interferant, for example, phenol-containing species. Some examples of chromatography-packing materials include Optipore L-493 (Dow Chemical Company, Providence, RI), SP-850 (Mitsubishi Chemical America, White Plains, NY), Amberlite XAD-4 (Rohm and Haas, Philadelphia, PA), and LC-18 (Supleco, Bellefonte, Pennsylvania).

In some embodiments, fused silica, Amberlite XAD-2, Amberlite IRC-50, Discovery DPA-6s, C-6 Bulk Phenyl, and other affinity-based packings or adsorbents synthesized from fused silica and/or TEOS with different phenyl derivatized silanes, can be used as the sorbents. In some embodiments, the sorbents are formed from carbon-based solids.

In some embodiments, sorbents are coated onto an inert support material, such as treated diatomaceous earth or other silica based materials (for example, solid silica support particles can have an organic coating bonded to their surface, wherein the bonding is produced by reacting a halogen substituted organosilane with the surface –OH groups present on the silica support). Generally, these coatings are non-polar in nature and therefore retention of the interfering species is produced by dispersion forces, making them useful for separation of organic compounds based on slight differences in their backbone or side chain configuration.

In some embodiments, the affinity domain can be manufactured using molecular imaging technology. In this embodiment, a sorbent is selected or prepared that is useful for binding a pre-determined interferant on the surface of a material by complementary functional group interaction. For example, a cross-linked styrene divinyl benzene material can be prepared that is imprinted with acetaminophen. U.S. Patents 5,453,199 and 5,872,198, both of which are incorporated by reference herein in their entirety, describe molecular imaging technology that can be used for imprinting acetaminophen or other interferants on the surface of a material. Complementary functional group interaction provides a selective, reversible association between the interferant and the material surface. Such methods for making binding surfaces are referred to hereinafter as "molecular imaging" methods and form surfaces referred to hereinafter as "imaged surfaces."

Molecular imaging provides a high surface area chromatography matrix material with molecular-specific sorbents. The imaged surfaces bind with interferants by covalently adhering, in a way that is geometrically controlled at least in the direction parallel, and preferably also in a direction normal to an underlying surface plane, a plurality of charged groups, hydrophobic groups, and various combinations thereof, to form a mirror image of groups complementary to them on a molecular surface of a target molecule, for example acetaminophen. These groups are preferably spaced about a hydrophilic undersurface rich in hydrogen containing groups and electronegative atoms such as oxygen, nitrogen, phosphorus, or sulfur that take part in formation of hydrogen bonds.

In some embodiments, a silica-like sol-gel material is imaged similarly to that described above with reference to molecular imaging. U.S. Patent 6,057,377, which is incorporated herein by reference in its entirety, describes a method for molecularly imprinting the surface of a sol-gel material, by forming a solution including a sol-gel material, a solvent, an imprinting molecule, and a functionalizing siloxane monomer of the form Si(OR)₃-n Xₙ, wherein n is an integer between zero and three and X is a functional group capable of reacting or associating with the imprinting molecule. In some embodiments, the phenyl silane bisphenyldimethylpropytrimethoxysilane, N-phenylaminopropyltrimethoxysilane, phenyldiethoxysilane, or phenyltriethoxysilane, for example.

The resulting sol-gel structure would include a three dimensional material imprinted with acetaminophen or other interferant. In this embodiment, the solvent is evaporated, and the imprinting molecule removed to form the molecularly imprinted sol-gel material. The removal of the imprinting molecule creates a pocket, which has the correct geometry and hydrogen binding to bind the interfering species as it passes through the structure. This sol-gel structure can then be ground using a mortar-pestle, or the like, and added to the membrane system as the affinity domain.

The use of sol-gel materials advantageously allow the material porosity, pore size, density, surface area, hardness, electrostatic charge, polarity, optical density, and surface hydrophobicity to be tailored to suit the affinity domain useful in the preferred embodiments.

### Experiment

An affinity domain of the preferred embodiments was prepared by blending chromatographic packings into a selected material and then cured. Particularly, chromatographic packings (Optipore L-493, Dow Chemical Company, Providence, RI) were ground and mixed 10% by weight with a polyurethane dispersion (Bayhydrol 123, Bayer, Pittsburgh, PA) and cast onto a carrier layer (ChronoThane^{TM} H, CM Biomaterials, Woburn, MA). This affinity domain was then laminated onto a membrane system including a resistance domain, enzyme domain, interference domain, and electrolyte domain such as described in U.S. Patent 6,001,067, which is incorporated herein by reference in its entirety. The membrane system was placed on glucose sensors such as described with reference to Fig. 1A, above, and glucose and acetaminophen tests were performed.

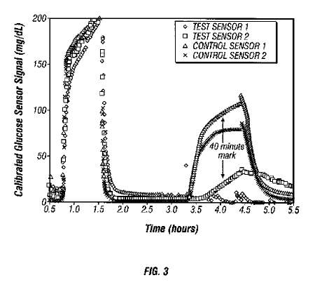

Fig. 3 is a graph of the response of test and control glucose sensors to glucose and acetaminophen standard step concentrations, including two glucose sensors each with a control membrane system and two glucose sensors each with a test multilayer membrane including an affinity domain of the preferred embodiments. The control sensors ("W55-4Layer/Lam-D100-123-Control") included a membrane system with a resistance domain, enzyme domain, interference domain, and electrolyte domain such as described in U.S. Patent 6,001,067. The test sensors ("W55-4Layer/Lam-D100-L493-123") included the control membrane system with an affinity domain laminated thereon. The affinity domain was prepared as described in this experiment. The x-axis represents time in hours. The y-axis represents calibrated glucose sensor signal strength in mg/dL.

Initially, the glucose sensors were placed in phosphate buffer and allowed to equilibrate for 15 minutes. Glucose was then added to a concentration of 200 mg/dL. The glucose sensor responses are shown on the graph by calibrated glucose sensor signals for each of the four sensors up to an approximate reading of 200 mg/dL after about one hour (Fig. 3, at t≈1.5), indicating functional glucose sensors. The solution was then changed to a buffer with a glucose concentration of 0 mg/dL and the calibrated sensor signals returned to approximately 0 mg/dL. After one and one-half hours in buffer (Fig. 3, at t≈3.5), acetaminophen was added to a concentration of 200 mM. After approximately one hour of exposure to the 200mM acetaminophen (Fig. 3, at t≈4.5), the control sensors showed calibrated glucose sensor signals up to about 75 to 120 mg/dL, indicating the sensitivity of the control sensors to acetaminophen as an interferant. However, the test sensors, including the affinity domain of the preferred embodiments, showed significantly reduced sensitivity to the acetaminophen concentration as compared to the control membranes (for example, test signals of less than about 40 mg/dL). After the hour of acetaminophen exposure (Fig. 3, at t≈4.5), the sensors were returned to buffer. The signal associated with the control sensors quickly returned back to zero, however the test sensors showed a slower return to zero signal strength.

These data show that the use of an affinity domain of the preferred embodiments provides significant dampening of the signal due to interferants as compared to membrane systems without the affinity domain. Thus, the release of the interferant from the affinity domain is sufficiently lowered and distributed over time, as compared to a membrane system without the affinity domain of the preferred embodiments, such that the effective local concentration of the interferant at the sensor head is below a level that can substantially interfere with sensor accuracy.

Methods and devices that are suitable for use in conjunction with aspects of the preferred embodiments are disclosed in co-pending U.S. Patent Application No. 10/885,476 filed July 6, 2004 and entitled "SYSTEMS AND METHODS FOR MANUFACTURE OF AN ANALYTE-MEASURING DEVICE INCLUDING A MEMBRANE SYSTEM"; U.S. Patent Application No. 10/842,716, filed May 10, 2004 and entitled, "MEMBRANE SYSTEMS INCORPORATING BIOACTIVE AGENTS"; co-pending U.S. Patent Application No. 10/838,912 filed May 3, 2004 and entitled, "IMPLANTABLE ANALYTE SENSOR"; U.S. Patent Application No. 10/789,359 filed February 26, 2004 and entitled, "INTEGRATED DELIVERY DEVICE FOR A CONTINUOUS GLUCOSE SENSOR"; U.S. Application No. 10/685,636 filed October 28, 2003 and entitled, "SILICONE COMPOSITION FOR MEMBRANE SYSTEM"; U.S. Application No. 10/648,849 filed August 22, 2003 and entitled, "SYSTEMS AND METHODS FOR REPLACING SIGNAL ARTIFACTS IN A GLUCOSE SENSOR DATA STREAM"; U.S. Application No. 10/646,333 filed August 22, 2003 entitled, "OPTIMIZED SENSOR GEOMETRY FOR AN IMPLANTABLE GLUCOSE SENSOR"; U.S. Application No. 10/647,065 filed August 22, 2003 entitled, "POROUS MEMBRANES FOR USE WITH IMPLANTABLE DEVICES"; U.S. Application No. 10/633,367 filed August 1, 2003 entitled, "SYSTEM AND METHODS FOR PROCESSING ANALYTE SENSOR DATA"; U.S. Patent No. 6,702,857 entitled "MEMBRANE FOR USE WITH IMPLANTABLE DEVICES"; U.S. Appl. No. 09/916,711 filed July 27, 2001 and entitled "SENSOR HEAD FOR USE WITH IMPLANTABLE DEVICE"; U.S. Appl. No. 09/447,227 filed November 22, 1999 and entitled "DEVICE AND METHOD FOR DETERMINING ANALYTE LEVELS"; U.S. Appl. No. 10/153,356 filed May 22, 2002 and entitled "TECHNIQUES TO IMPROVE POLYURETHANE MEMBRANES FOR IMPLANTABLE GLUCOSE SENSORS"; U.S. Appl. No. 09/489,588 filed January 21, 2000 and entitled "DEVICE AND METHOD FOR DETERMINING ANALYTE LEVELS"; U.S. Appl. No. 09/636,369 filed August 11, 2000 and entitled "SYSTEMS AND METHODS FOR REMOTE MONITORING AND MODULATION OF MEDICAL DEVICES"; and U.S. Appl. No. 09/916,858 filed July 27, 2001 and entitled "DEVICE AND METHOD FOR DETERMINING ANALYTE LEVELS," as well as issued patents including U.S. 6,001,067 issued December 14, 1999 and entitled "DEVICE AND METHOD FOR DETERMINING ANALYTE LEVELS"; U.S. 4,994,167 issued February 19, 1991 and entitled "BIOLOGICAL FLUID MEASURING DEVICE"; and U.S. 4,757,022 filed July 12, 1988 and entitled "BIOLOGICAL FLUID MEASURING DEVICE"; U.S. Appl. No. 60/489,615 filed July 23, 2003 and entitled "ROLLED ELECTRODE ARRAY AND ITS METHOD FOR MANUFACTURE"; U.S. Appl. No. 60/490,010 filed July 25, 2003 and entitled "INCREASING BIAS FOR OXYGEN PRODUCTION IN AN ELECTRODE ASSEMBLY"; U.S. Appl. No. 60/490,009 filed July 25, 2003 and entitled "OXYGEN ENHANCING ENZYME MEMBRANE FOR ELECTROCHEMICAL SENSORS"; U.S. Appl. No. 10/896,312 filed July 21, 2004 and entitled "OXYGEN-GENERATING ELECTRODE FOR USE IN ELECTROCHEMICAL SENSORS"; U.S. Appl. No. 10/896,637 filed July 21, 2004 and entitled "ROLLED ELECTRODE ARRAY AND ITS METHOD FOR MANUFACTURE"; U.S. Appl. No. 10/896,772 filed July 21, 2004 and entitled "INCREASING BIAS FOR OXYGEN PRODUCTION IN AN ELECTRODE ASSEMBLY"; U.S. Appl. No. 10/896,639 filed July 21, 2004 and entitled "OXYGEN ENHANCING ENZYME MEMBRANE FOR ELECTROCHEMICAL SENSORS"; U.S. Appl. No. 10/897,377 filed July 21, 2004 and entitled "ELECTROCHEMICAL SENSORS INCLUDING ELECTRODE SYSTEMS WITH INCREASED OXYGEN GENERATION". The foregoing patent applications and patents are incorporated herein by reference in their entireties.

All references cited herein are incorporated herein by reference in their entireties. To the extent publications and patents or patent applications incorporated by reference contradict the disclosure contained in the specification, the specification is intended to supersede and/or take precedence over any such contradictory material.

The term "comprising" as used herein is synonymous with "including," "containing," or "characterized by," and is inclusive or open-ended and does not exclude additional, unrecited elements or method steps.

All numbers expressing quantities of ingredients, reaction conditions, and so forth used in the specification and claims are to be understood as being modified in all instances by the term "about." Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

1. A membrane suitable for use with an analyte sensor, the membrane comprising an affinity domain, wherein the affinity domain comprises a sorbent having an affinity for an interfering species.

2. The membrane of claim 1, wherein the sorbent has an affinity for a phenol-containing species.

3. The membrane of claim 2, wherein the sorbent has an affinity for acetaminophen.

4. The membrane of claim 1, wherein the sorbent comprises an adsorbent substance.

5. The membrane of claim 4, wherein the adsorbent substance comprises a chromatography-packing material.

6. The membrane of claim 1, wherein the sorbent comprises a molecularly imprinted surface adapted to bind with the interfering species by covalent adherence.

7. The membrane of claim 1, wherein the sorbent comprises a molecular structure that has a geometric structure and hydrogen binding capability, wherein the molecular structure is adapted to bind with the interfering species.

8. An electrochemical sensor comprising the membrane of claim 1.

9. A wholly implantable glucose sensor comprising the membrane of claim 1.

10. A transcutaneous glucose sensor comprising the membrane of claim 1.

11. An analyte sensor for measuring the concentration of an analyte in a host, the sensor comprising:
a sensing region for sensing the analyte; and
a membrane system comprising an affinity domain, the affinity domain having an affinity for an interfering species, wherein the membrane system is disposed adjacent to the sensing region.

12. The sensor of claim 11, wherein the sensing region comprises an electroactive surface and wherein the membrane system comprises an enzyme capable of reacting with the analyte.

13. The sensor of claim 11, wherein the affinity domain comprises a sorbent, wherein the sorbent is configured to slow the diffusion of the interfering species through the membrane system to the sensing region.

14. The sensor of claim 13, wherein the sorbent has an affinity for a phenol-containing species.

15. The sensor of claim 14, wherein the sorbent has an affinity for acetaminophen.

16. The sensor of claim 11, wherein the sensor is adapted for implantation in a soft tissue of the host.

17. The sensor of claim 16, wherein the sensor is adapted for whole implantation within the host.

18. The sensor of claim 16, wherein the sensor is adapted for transcutaneous implantation in the host.
